(19) United States
(12) Patent Application Publication
(10) Pub. No.: US 2025/0302710 A1
(43) Pub. Date: Oct. 2, 2025

(54) SKIN CARE COMPOSITIONS CONTAINING A RETINOID WITH REDUCED MALODOR

(71) Applicant: The Procter & Gamble Company, Cincinnati, OH (US)

(72) Inventors: Kavya Narayanan, Cincinnati, OH (US); Shawn David McConaughy, Cincinnati, OH (US); Susan Eleanor Baillely, Cincinnati, OH (US); Laura Lynn McElroy, Okeana, OH (US); Natalia Lebedev, Mason, OH (US); Liu Ping Adachi, Singapore (SG); Yeow Pei San, Singapore (SG)

(21) Appl. No.: 19/092,086

(22) Filed: Mar. 27, 2025

Related U.S. Application Data
(60) Provisional application No. 63/570,292, filed on Mar. 27, 2024.

(51) Int. Cl. A61K 8/33; A61K 8/67; A61Q 19/02; A61Q 19/08

(52) U.S. Cl. CPC A61K 8/33; A61Q 19/02; A61Q 19/08; A61K 8/671; A61K 2800/30; A61K 2800/40

(57) ABSTRACT

A skin care composition having reduced malodor. The skin care composition includes a retinoid and an aldehyde in a dermatologically acceptable carrier. The aldehyde can be an unsaturated cyclic aldehyde with 11 carbon atoms and/or a mixture of linear aldehydes having a chain length from about 4 to about 20 carbon atoms, alternatively from about 8 to 11 carbon atoms.

### FIELD OF THE INVENTION

The present disclosure generally relates to a stable skin care composition containing a retinoid. More specifically, the present invention relates to a skin care composition in the form of an oil-in-water emulsion containing a retinoid with reduced malodor.

### BACKGROUND OF THE INVENTION

Skin care products can enhance the health and/or appearance of a user's skin. Presently, there is a diverse array of topical skincare products available, specifically formulated for acne management and anti-aging purposes. Many of these products aim to delay, minimize, or eliminate common signs of skin aging, including wrinkles, discoloration, and other histological changes induced by environmental damage or the natural aging process to restore a more youthful and/or vibrant appearance.

Many skin care products, including moisturizers and serums, can include vitamins, vitamin derivatives, and/or other active ingredients for improving skin appearance. For example, vitamin A, also referred to as retinol, is known for use in topical skin care compositions to provide skin health and/or appearance benefits. Vitamin A, along with its derivatives, form a class of compounds commonly referred to as “retinoids.” At one time, retinoids were primarily used for the treatment of acne. More recently, retinoids have also been used in the treatment of photo- and/or intrinsically aged skin.

However, formulating stable skin care products containing a retinoid with an acceptable shelf life (e.g., 2+ years, preferably 3+ years) can be challenging because retinoids are undesirably reactive and susceptible to degradation. Some known sources of degradation include oxidation, light exposure (e.g., ultraviolet radiation), heat (e.g., temperatures of 40° C. or more), and interactions with other ingredients in the formula. As retinoids degrade, they are less efficacious and the composition can yellow and it can have a noticeable malodor, reminiscent of sour milk.

Furthermore, some retinol products may include a preservative and other ingredients that are intended to boost retinoid stability that are free from formaldehyde-releasing agents. However, over time, the preservative, as well as the ingredients intended to enhance the stability of retinoids, may develop a malodor. This can further contribute to the overall malodor of the formula.

Accordingly, it would be desirable to provide a retinoid-containing skin care composition with reduced malodor. Particularly, a retinoid-containing skin care composition with reduced malodor and a preservative system that is free from formaldehyde-releasing agents.

### SUMMARY OF THE INVENTION

A skin care composition having reduced malodor comprising: (a) an unsaturated cyclic aldehyde with at least 11 carbon atoms; (b) a retinoid; (c) a dermatologically acceptable carrier.

A skin care composition having reduced malodor comprising: (a) a mixture of linear aldehydes comprising: (i) a first linear aldehyde comprising a chain length of about 4 to about 20 carbon atoms; (ii) a second linear aldehyde comprising a chain length of about 4 to about 20 carbon atoms; wherein the chain length of the first aldehyde and the second aldehyde are different; (b) a retinoid; (c) a dermatologically acceptable carrier.

A skin care composition having reduced malodor comprising: (a) a malodor reducing material comprising an aldehydic moiety; (b) a retinoid; (c) a dermatologically acceptable carrier.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows discrete datapoints and a best fit line for the odor grade over ˜3 years for Olay® Retinol24 Moisturizer and Example 1 of storage at ambient conditions;

FIG. 2 shows discrete datapoints and a best fit line for the odor grade over ˜3 years for Olay® Retinol24 Moisturizer and Example 1 of storage at 40° C. for ˜6 months.

### DETAILED DESCRIPTION OF THE INVENTION

Retinoids are a class of compounds derived from Vitamin A that are frequently incorporated into skin care compositions for anti-wrinkle, acne, and hyperpigmentation benefits. It can be challenging to formulate with retinoids because they have a high propensity for degradation. Retinoids are sensitive to oxidation, hydrolysis, heat, and UV. As retinoids degrade, there is less active available to penetrate the skin and the skin care product becomes less effective. In addition, the formula can be noticeably more yellow and can have an off-putting smell, reminiscent of sour milk.

In response to the demands of some consumer and regulatory agencies, formaldehyde-releasing agents may be removed from skin care products. For example, a preservative system that previously included DMDM hydantoin (commercially available as Glydant®) and DMDM hydantoin and iodopropynyl butylcarbamate (commercially available as Glydant® Plus Liquid) has been replaced with a formaldehyde-free alternative. The new preservative system can incorporate a cell wall disrupter, such as phenoxyethanol and hydroxy acetophenone (commercially available as Symsave® H from Symrise®), along with an antioxidant like carnosine. This change ensures compliance with the elimination of formaldehyde-releasing agents while maintaining the product's stability and effectiveness.

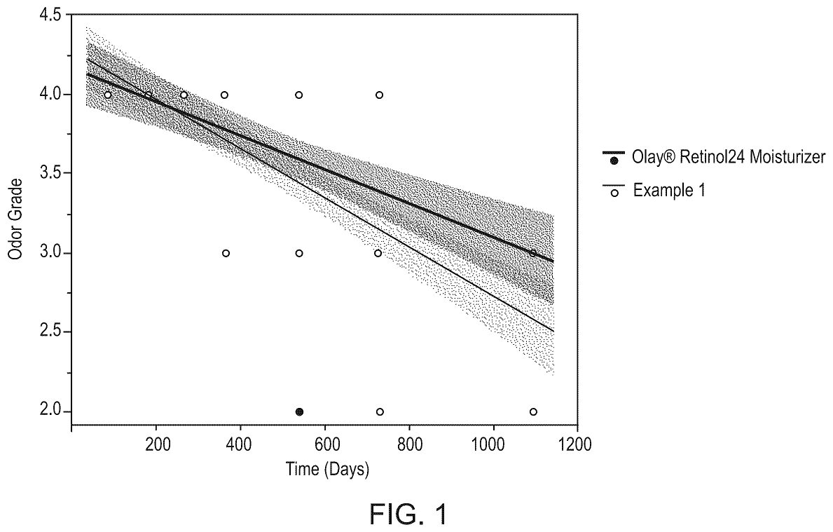

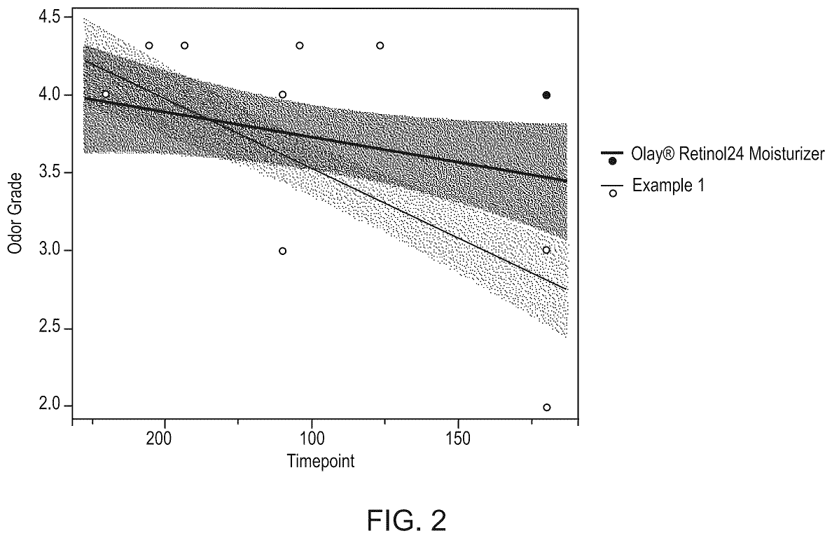

Qualified odor evaluators graded samples of Olay® Retinol24+Peptide Night Face Moisturizer that includes retinol, retinyl propionate, and DMDM hydantoin preservative (LOT2081TN42) and Example 1 in Table 1, below, that includes retinyl propionate, preservatives, and retinoid stability boosters with phenoxyethanol, hydroxyacetophenone, carnosine, and sodium sulfite. The Olay® Retinol24 Moisturizer was purchased ˜17 months after it was made. Therefore, at the start of the assessment (day 0), these samples had already been subjected to a 17-month aging period under typical conditions experienced during shipping, warehousing, and while being displayed on a store shelf. The odor evaluators graded each sample on a scale from 0 to 5 (integer values only), where a rating of 5 represented the most favorable (i.e., no malodor present). If the score was 3 or above, it indicated that the composition was considered acceptable to consumers. However, even if a composition is deemed consumer acceptable, there is still a possibility of a noticeable malodor being present. The Olay® Retinol24 Moisturizer and Example 1 were each tested 13 times at each condition. The individual scores were combined and plotted to generate a curve representing the odor grades over time. The resulting curve, depicted in FIGS. 1 and 2, was generated using a model that determined the best fit. Note, there are fewer than 13 visible points in both FIGS. 1 and 2 due to overlapping data points.

FIG. 1 shows samples at ambient temperature over ˜3 years and FIG. 2 shows the samples subjected to accelerated stability conditions of 40° C. for ˜6 months. The best fit curve in both FIGS. 1 and 2 shows that both retinol products have more malodor over time. However, there is a general trend that Example 1, which includes phenoxyethanol, hydroxyacetophenone, sodium sulfite, and carnosine, had a lower odor grade, indicating that the inclusion of ingredients to improve formula stability, including microhostility and retinoid stability may have contributed to an increase in the malodor severity.

It was found that certain aldehydes can reduce the malodor from retinoid degradation and can also help mitigate the malodor from other ingredients in the formulation. Table 5, hereafter, summarizes the fragrance evaluation by three trained perfume experts. The examples that included certain aldehydes, in particular the 6-cyclopentylidenchexanal or the linear aldehydes with a chain length of 8 to 11 carbon atoms, reduced malodor, as compared to the control.

The aldehydes can be included at a level to block malodor, but low enough that a consumer cannot perceive the presence of the aldehyde in the formulation. The composition can include from about 0.00005% to about 0.05% of the aldehydes, alternatively from about 0.0001% to about 0.05%, alternatively from about 0.0002% to about 0.05%. The composition can include from about 0.00005% to about 0.01% of the aldehydes, alternatively from about 0.0001% to about 0.0005%, alternatively from about 0.0001% to about 0.0005%, alternatively from about 0.0002% to about 0.0005%, alternatively from about 0.00025% to about 0.0025%, and alternatively from about 0.0002% to about 0.002%. The composition can include from about 0.00005% to about 0.05% of the aldehydes, alternatively from about 0.0001% to about 0.04%, alternatively from about 0.0002% to about 0.03%, alternatively from about 0.0004% to about 0.02%, and alternatively from about 0.0005% to about 0.01%.

An example of an aldehyde that was an effective odor blocker is an unsaturated cyclic aldehyde with at least 8 carbon atoms, alternatively at least 9 carbon atoms, alternatively at least 10 carbon atoms, alternatively at least 11 carbon atoms, particularly where an unsaturated cyclic aldehyde with 11 carbon atoms the carbon atoms can be in both cyclic and linear configuration such as 6-cyclopentylidenchexanal. The cyclic portion can be saturated. The cyclic portion can have 5-7 members. The linear chain can have one degree of unsaturation. The linear chain can have from about 7 carbon atoms to about 20 carbon atoms, alternatively from about 9 carbon atoms to about 15 carbon atoms, and alternatively from about 10 carbon atoms to about 13 carbon atoms.

Another example of an aldehyde that was an effective odor blocker is a combination of linear aldehydes with a chain length of 4 to 20 carbon atoms (C4-C20), alternatively from 6 to 16 carbon atoms (C6-C16), alternatively from 7 to 14 carbon atoms (C7-C14), and alternatively from 8 to 11 carbon atoms (C8-C11). The linear chain can be saturated. In some examples, only one aldehyde with a certain chain length is purposefully added. In another example, a mixture of aldehydes with different chain lengths is purposefully added. The mixture can include a first aldehyde with a first carbon chain length, a second aldehyde with a second carbon chain length, optionally a third aldehyde with a third carbon chain length, and optionally a fourth aldehyde with a fourth carbon chain length. The first, second, third, and fourth chain lengths can all have different carbon chain lengths. The mixture can include the first aldehyde, second aldehyde, third aldehyde (if present), and fourth aldehyde (if present) at ≥10%, by weight of the mixture, alternatively ≥15%, by weight of the mixture, alternatively ≥17%, by weight of the mixture, and alternatively ≥20%, by weight of the mixture. The mixture can include the first aldehyde, second aldehyde, third aldehyde (if present), and fourth aldehyde (if present) at ≤45%, by weight of the mixture, alternatively ≤40%, by weight of the mixture, alternatively ≤35%, by weight of the mixture, and alternatively ≤30%, by weight of the mixture. The first, second, third, and/or fourth aldehyde can be present at a weight ratio of about 1:2 to about 2:1 relative to another aldehyde in the composition, alternatively from about 2:3 to about 4:2, alternatively from about 3:4 to about 4:3, and alternatively from about 4:5 to about 5:1.

### Composition

The skin care composition can be a single phase, or it can be an emulsion that includes a continuous phase and a dispersed phase. The skin care composition can include a carrier, such as water, polymeric thickening agents, antioxidants, a preservative system, oils, including silicone oils, retinoids, and the odor blocking technology described herein.

The dermatologically acceptable carrier enables other components (e.g., actives like retinoids) to be delivered to the skin at an appropriate concentration. The carrier can thus act as a diluent, dispersant, solvent, or the like for particulate material, which helps ensure that it can be applied to and distributed evenly over the selected target at an appropriate concentration. The carrier may contain one or more dermatologically acceptable solid, semi-solid or liquid fillers, diluents, solvents, extenders and the like. The carrier may be solid, semi-solid, or liquid. In some instances, the carrier can be inert or it can provide benefits of its own to keratinous tissue. Concentrations of the carrier can vary with the carrier selected and the intended concentrations of the composition components.

The type of carrier utilized in the present skin care composition depends on the type of product form desired for the composition. The topical composition can be any cosmetic composition, such as moisturizers including lotions and creams, serums, gels, sticks, ointments, pastes, and mousses. These product forms may comprise several types of carriers including, but not limited to, solutions, aerosols, emulsions, gels, solids, and liposomes.

The skin care composition can be free of or formulated without formaldehyde and formaldehyde-releasing agents. The skin care composition can be free of DMDM hydantoin, imadazolidinyl urea, diazlidinyl urea, sodium hydroxyl, methyl glycinate, and combinations thereof. The skin care composition can also be free of parabens and/or sulfate-based surfactants including SLS and SLES.

The composition can have a b-value of less than 25, less than 20, less than 18, less than 14, alternatively less than 13, alternatively less than 12, alternatively less than 11, and alternatively less than or equal to 10 according to the Color Test Method, described hereafter.

The retinol composition can include a preservative system that acts as a cell wall disrupter, as described below, in combination with an antioxidant, and a reducing agent. The retinol composition can include a preservative system comprising hydroxyacetophenone, an antioxidant comprising carnosine, and a reducing agent comprising sodium sulfite as shown and exemplified in U.S. patent application Ser. No. 18/460,804.

### Preservative System

As used herein, a preservative system can be one or more substances and/or chemicals that is added to cosmetic compositions that can prevent decomposition by microbial growth and/or by undesirable chemical changes. The preservative system can be free of formaldehyde and formaldehyde-releasing agents. In some examples, the preservative system can contain one or more ingredients that are registered as preservatives by the European Chemicals Agency as of Sep. 27, 2022 (hereinafter ECA). In other examples, the preservative system can contain one or more ingredients that are not registered as preservatives by the ECA. Alternatively, the preservative system can be free of ingredients that are registered as preservatives by the ECA.

The composition can contain from about 0.01% to about 1.5%, alternatively from about 0.05% to about 1.25%, alternatively from about 0.1% to about 1% of the preservative system, alternatively from about 0.2% to about 0.8%, and alternatively from about 0.3% to about 0.5%.

The preservative system can include one or more cell wall disrupters, which can inhibit microbial growth by disrupting microbial cell walls. Non-limiting examples of cell wall disrupters can include aromatic alcohols, mid chain diols, organic acids, hydroxyacetophenone, 1,2-Hexanediol (and) Caprylyl Glycol, ethylhexylglycerin, methylheptylglycerin, or a combination thereof.

Non-limiting examples of aromatic alcohols can include phenoxyethanol, tryptophol, tyrosol, phenethyl alcohol, benzyl alcohol, or a combination thereof.

Non-limiting examples of mid chain diols can include caprylyl glycol, pentylene glycol, hexylene glycol, or a combination thereof.

Non-limiting examples of organic acids can include benzoic acid, sorbic acid, levulinic acid, and salicylic acid, or a combination thereof.

### Chelating Agent

The cosmetic compositions can include from about 0.05% to about 0.5% chelating agent, and alternatively from about 0.1% to about 0.3%. As used herein, “chelating agent” means an active agent capable of removing a metal ion from a system by forming a complex so that the metal ion cannot readily participate in or catalyze chemical reactions. In addition, chelating agents can be used in combination with the preservative system described herein to boost the efficacy of the preservative. Non-limiting examples of chelating agents can include EDTA (disodium EDTA, tetrasodium EDTA), tetrahydroxypropyl ethylenediamine, etidronic acid, sodium phytate, phytic acid, oxalic acid and derivatives, sodium gluconate, EDDS (trisodium ethylenediamine disuccinate), or a combination thereof.

### Antioxidant

The cosmetic compositions can include from about 0.001% to about 1% antioxidant, alternatively from about 0.005% to about 0.85%, alternatively from about 0.01% to about 0.75%, alternatively 0.05% to about 0.5%, and alternatively from about 0.1% to about 0.3%.

The antioxidant can be a non-vitamin antioxidant. Non-limiting examples of antioxidants can include hydroxycinnamic acid, ferulic acid, bakuchiol, tea extracts including Camellia sinensis leaf extract (white tea) and green tea extract, caffeine, Rosmarinus officinalis leaf extract, Octadecyl Di-t-butyl-4-hydroxyhydrocinnamate, carnosine, Ethylene bis(oxyethylene) bis-(3-(5-tert-butyl-4-hydroxy-m-tolyl) propionate), Punica fruit granatum extract, butylated hydroxytoluene (BHT), L-ergothioneine (available as THIOTANE™), tetrahydrocurcumin, cetyl pyridinium chloride, diethylhexyl syrinylidene malonate (available as OXYNEX™), hexadec-8-ene-1,16-dicarboxylic acid (octadecene dioic acid; ARLATONE™ Dioic DCA from Uniqema), ubiquinone (co-enzyme Q10), yeast extracts or yeast culture fluid (e.g., Piterao), or a combination thereof.

The antioxidant can be a vitamin antioxidant. Non-limiting examples of vitamin antioxidants can include vitamin C, B vitamins including niacinamide, vitamin D, vitamin E, vitamin K, or a combination thereof.

### Reducing Agents

One or more reducing agents can be added to the composition to help mitigate yellowness. The composition can include from about 0.01% to about 0.5% reducing agent, alternatively from about 0.02% to about 0.2%, and alternatively from about 0.05% to about 0.1%.

Non-limiting examples of reducing agents can include sulfite salts, acetyl farnesylcysteine, aminoethanesulfinic acid, butyrolactonethiol, ethanolamine dithiodiglycolate, ethyl thioglycolate, formamidine sulfinic acid, hydrolyzed saccharomyces/lactobacillus/ubiquinone ferment, sodium glyoxylate, sodium hydroxymethane sulfonate, sodium oxymethylene sulfoxylate, sodium thioglycolate, sodium thiosulfate pentahydrate, sodium thiosulfate pentahydrate, potassium sulfite, sodium bisulfite, sodium metabisulfite, sodium thiosulfate, potassium metabisulfite, or a combination thereof.

Non-limiting examples of sulfite salts include sodium sulfite, potassium sulfite, ammonium bisulfite, ammonium sulfite, potassium metabisulfite, potassium sulfite, sodium bisulfite, sodium hydrosulfite, sodium metabisulfite, or a combination thereof.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 15%, alternatively from about 3% to about 10%, alternatively from about 4% to about 9%, and alternatively from about 5% to about 8% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 40% by weight of composition (e.g., 1% to 35%, 2% to 30%, 3% to 25%, 4% to 20%, or even 5% to 18%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., Mw of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Thickening Agents

The composition may include one or more thickening agents, alternatively at least two thickening agents. In some examples, the composition is a moisturizer and can contain from about 0.5% to about 6% thickening agent, alternatively from about 0.75% to about 5%, and alternatively from about 1% to about 4%. In other examples, the composition is a serum and can contain from about 0.1% to about 4% thickening agent, alternatively from about 0.25% to about 3%, alternatively from about 0.5% to about 2%. Suitable classes of thickening agents include but are not limited to carboxylic acid polymers, polyacrylamide polymers, sulfonated polymers, copolymers thereof, hydrophobically modified derivatives thereof, and mixtures thereof.

The composition can include a carboxylic acid polymer thickening agent such as a carbomer. The composition can contain from about 0.1% to about 1%, alternatively from about 0.2% to about 0.8%, alternatively 0.25% to about 0.7%, alternatively from about 0.3% to about 0.6%, and alternatively from about 0.4% to about 0.5% of a carboxylic acid polymer thickening agent.

The composition can include a polyacrylamide polymer and copolymer thickening agent. The composition can contain from about 0.8% to about 3%, alternatively from about 0.9% to about 2.5%, alternatively from about 1% to about 2.2%, alternatively from about 1.1% to about 2.1%, alternatively from about 1.2% to about 1.9%, or alternatively from about 1.3% to about 1.8% polyacrylamide polymer and copolymer thickening agent.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-30 alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic® Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic® Corp. and Viscolam® at 100 P available from Lamberti S.p.A. (Gallarate, Italy). Another commercially available material comprising a sulfonated polymer is Sepiplus™ 400 available from Seppic® Corp.

Further, suitable thickening agents may include superabsorbent polymers. These superabsorbent polymers may be chosen from: crosslinked sodium polyacrylates, such as, for example, those sold under the names Octacare X100, X110 and RM100 by Avecia®, those sold under the names Flocare GB300 and Flosorb 500 by SNF™, those sold under the names Luquasorb 1003, Luquasorb 1010, Luquasorb 1280 and Luquasorb 1100 by BASF®, those sold under the names Water Lock G400 and G430 (INCI name: Acrylamide/Sodium Acrylate Copolymer) by Grain Processing®, or Aqua Keep® 10 SH NF, Aqua Kecp® 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries®, Makimousse 12 and Makimouse 25 supplied by Kobo Products Inc (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing® (INCI name: Starch/Acrylamide/Sodium Acrylate Copolymer). Preferred superabsorbent polymers can include Makimousse 12 and Makimousse 25.

Suitable thickening agents for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob trec; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

### Retinoid

The skin care compositions can include a safe and effective amount of a retinoid. As used herein, “retinoid” includes all natural and/or synthetic analogs of Vitamin A or retinol or retinol-like compounds which possess the biological activity of Vitamin A in the skin, as well as the geometric isomers and stereoisomers of these compounds. For example, the retinoid may be a retinol ester (e.g., C2-C22 alkyl esters of retinol, including retinyl palmitate, retinyl acetate, and retinyl propionate), retinol aldehydes, retinal, beta-carotene, and/or retinoic acid (including all-trans retinoic acid and/or 13-cis-retinoic acid). A particularly suitable example of a retinoid for use in the present composition is retinyl propionate (“RP”). These compounds are well known in the art and are commercially available from a number of sources, e.g., Sigma Chemical Company (St. Louis, MO), Boerhinger Mannheim (Indianapolis, IN), BASF (Mt. Olive, NJ), and Roche (Basel, Switzerland). Other suitable retinoids are tocopheryl-retinoate (tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]-nicotinate). The retinoid may be included as a pure or substantially pure material, or as an extract obtained by suitable physical and/or chemical isolation from natural (e.g., plant) sources.

The composition may contain 0.0001% to 2% (e.g., 0.005% to 2%, 0.01% to 1%, 0.01% to 0.5%, 0.1% to 0.4%, 0.15% to 0.3%) of the retinoid. In some instances, mixtures of more than one retinoid may be used.

The retinoid present in the skin care compositions herein can be stable. In order for the skin care composition to provide the desired health or appearance benefit, it can be important to provide a suitable amount of retinoid active.

The composition can have ≥70% RP remaining, alternatively ≥65% RP remaining, alternatively ≥60% RP remaining, alternatively ≥55% RP remaining, alternatively ≥50% RP remaining at any time during the shelf life of the product. The % RP remaining is determined using the HPLC Method, described hereafter. The initial RP is the concentration of RP at the time of manufacture, which can be the % RP claimed on the product packaging or other materials (e.g., website, SmartLabel®, etc.).

### Silicone Oil

The composition can include a silicone oil selected from volatile silicone oil, non-volatile silicone oil, and combinations thereof. The silicone oil can be in the dispersed phase. The composition can include from about 1% to about 35% silicone oil, alternatively from about 5% to about 30% silicone oil, and alternatively from about 10% to about 28% silicone oil.

### Volatile Silicone Oil

Suitable volatile silicones can include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 cst, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

### Non-Volatile Silicone Oils

Suitable non-volatile silicone oils include polysiloxanes. Non-volatile polylsiloxanes may have a viscosity of from about 10 to about 1,000,000 centistokes at 25° C. Such polysiloxanes can be represented by the general chemical formula:

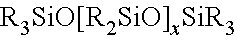

wherein each R is independently selected from hydrogen or C1-30 straight or branched chain, saturated or unsaturated alkyl, phenyl or aryl, trialkylsiloxy; and x is an integer from 0 to about 10,000. In certain embodiments, R is methyl or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids). Suitable dimethicones include those represented by the chemical formula:

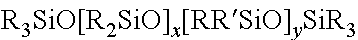

wherein R and R′ are each independently hydrogen or C1-30 straight or branched chain, saturated or unsaturated alkyl, aryl, or trialkylsiloxy; and x and y are each integers of 1 to 1,000,000. Examples include alkyl dimethicones wherein at least R′ is a fatty alkyl (e.g., C12-22). A suitable alkyl dimethicone is cetyl dimethicone, wherein R′ is a straight C16 chain and R is methyl, commercially available as 2502Cosmetic Fluid from Dow Corning.

Preferred non-volatile oils include dimethicones (polydimethylsiloxanes), preferably with viscosities of between 10 cst and 1000 est, more preferably between 15 cst to 400 cst, most preferably between 20 cst and 200 est. The average chain lengths for these preferred dimethicone materials is from about 12 to about 375 dimethylsiloxane units, more preferably from about 20 to about 200 dimethylsiloxane units, and most preferably with average chain lengths of from about 27 to about 125 dimethylsiloxane units. In one embodiment, the second composition will comprise at least one non-volatile silicone oil. In one such embodiment, at least about 70%, by weight of the non-volatile oil, is a non-volatile silicone oil. In another embodiment, at least about 80%, by weight of the nonvolatile oil, is a non-volatile silicone. In yet another embodiment, at least about 90%, by weight of the non-volatile oil, is a non-volatile silicone oil.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. The other optional ingredients can be present in the continuous phase and/or dispersed phase. Some nonlimiting examples of additional actives include additional vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance and/or an essential oil, or a colorant, in particular a natural colorant. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition. The optional ingredient, particularly the fragrance, can be included at amounts of from about 0.0001% to about 0.25%, alternatively from about 0.001% to about 0.1%, alternatively from about 0.005% to about 0.1%, and alternatively from about 0.01% to about 0.1%.

### Method of Use

The skin care composition may be applied to the face, neck, and/or a portion or combination thereof at least once a day, twice a day, or on a more frequent daily basis, during a treatment period. When applied twice daily, the first and second applications are separated by at least 1 to 12 hours. Typically, the composition is applied in the morning and/or at night before bed. The treatment period herein is ideally of sufficient time for the retinoids and/or other skin care actives to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The skin care composition can visibly improve fine lines and wrinkles, smoothness, brightness, firming, dark spots, and pores. The skin care composition can also prevent breakouts, blackheads, whiteheads, blemishes, and clogged pores. The skin care composition can restore skin tone and texture by clearing acne. The skin care composition can leave skin feeling hydrated and replenished. The skin care composition can be applied at night. The skin care composition can even skin texture and minimizes the appearance of pores.

### Definitions

As used herein, “formulated without” means that the ingredient is not intentionally added. However, “formulated without” does not guarantee “100% free from” since trace contaminants are possible.

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism). In some examples, the skin care active comprises one or more retinoids.

As used herein, “skin care composition” means a composition that includes a skin care active and regulates and/or improves skin condition.

It is to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

The term “about” is used herein to indicate a difference of up to ±10% from the stated number, such as ±9%, ±8%, ±7%, ±6%, ±5%, ±4%, ±3%, ±2%, or ±1%.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Test Methods

### Color Test Method

For the purposes of the present invention, color is defined according to a value on the CIELAB color system, which is based on XYZ color system, defined by the Commission Internationale de l'Eclairage (CIE system) to provide a manner of objectively representing perceived color and color differences. X, Y, and Z can be expressed in a variety of manners, or “scales,” one of which is the Hunter Scale. The Hunter scale has three variables, L, a, and b, which correlate mathematically to X, Y, and Z, and is described by Robertson, A. R. in “The CIE 1976 Color Difference Formulas,” Color Research Applications, vol. 2, pp. 7-11 (1977).

To measure the color of the compositions of the present invention, approximately 8 mL of the product to be tested is transferred into a petri dish (35×10 mm disposable) using a syringe. The Petri dish is then placed on the black half of a standard opacity chart (Form N2A, Leneta Company of Manwah, NJ or the equivalent thereof, of which the top half is black and the bottom half is white). The color (L, a, and b values) of the product in the petri dish is then measured using an integrated sphere spectrophotometer with settings selected to exclude specular reflection. The value for “a” correlates to a value along the red-green (horizontal) axis, and the value for “b” correlates to a value along the blue-yellow (vertical) axis. For example, a yellow-colored sample will have a positive b-value, whereas a green colored sample will have a negative a-value. A more positive or negative value represents a more intense color. The value for “L” is an indicator of lightness and/or darkness, and correlates to a value along the z-axis, which is perpendicular to both the horizontal and vertical axes.

### HPLC Test Method

This method provides a suitable means for determining the amount of RP loss in a composition.

A sufficient quantity of the test composition is placed in a controlled environment chamber/room at the desired temperature, humidity, and duration. When accelerated aging is complete, the sample is removed from the controlled environment, equilibrated to room temperature (21° C.±2° C.) for 24 hours, and then measured for chemical analysis shortly after. Note that retinoids are light sensitive and should not be exposed to direct light. All test compositions are mixed thoroughly before sampling.

The amount of RP loss is determined on a % w/w basis by HPLC (isocratic elution) as follows. The HPLC column(s) is conditioned in accordance with conventional practices.

### Chromatographic Conditions

1. Column: C18 (5 micron), 250 mm×4.6 mm
2. Mobile Phase (Eluent): methanol/2-propanol (70/30 v/v)
3. Column temperature: approximately 25° C. (Ambient)
4. UV wavelength: 280 nm
5. Injection volume: 20 microliters
6. Flow rate: 1.0 ml/min
7. Run time: approximately 22.0 minutes
8. RP retention time: 6 minutes

### Mobile Phase Preparation

Prepare 1 L of Mobile Phase (Eluent) by combining 700 mL of methanol with 300 mL of 2-propanol.

### External Standard Preparation (Prepare Fresh on Day of Use)

In a 25 ml amber flask, dissolve 25 mg of retinyl propionate in 10 ml of Mobile Phase. Avoid exposing the RP to light. Dilute to volume with Mobile Phase. Transfer a 5.0 ml aliquot to a 50 ml amber flask and dilute to volume with mobile phase. Mix well. Filter about 1 ml of the external standard thru a suitable 0.45 micron filter (e.g., Whatman GD/X) into an amber HPLC sample vial.

### HPLC Sample Preparation

Using a 1-cc tuberculin syringe, weigh and transfer 500 mg of the sample composition into a 25 ml amber volumetric flask. Add 10 mL of Mobile Phase and vortex on high for 2 minutes or until product is completely dispersed. Dilute to volume with mobile phase and mix well. Filter approximately 1 ml into an auto-sampler vial using a syringe filter (e.g., Whatman GD/X filter unit). Perform 20 injections using the condition described.

$$( A ) / ( B ) \times ( B ) / ( W ) \times ( DF ) \times 100 = retinyl propionate , \% w / w$$

Where,
A=peak area of retinyl propionate for the sample
B=peak area of retinyl propionate for the calibration
C=retinyl propionate standard weight in mg
W=sample weight, mg
DF=Dilution Factor (e.g., 0.1)

### Viscosity Test Method

After storing for at least 24 hours at 25° C., the product viscosity is measured by a DV2T-RV viscometer (available from Brookfield Engineering Laboratories, Middleboro, Massachusetts) with a TC spindle rotated at 5 rpm at 25° C. The spindle starts 2.5-3.0 cm below the product surface and travels a helipath downward and collects one reading every 9 seconds for 5 readings. The average (mean) of the readings is calculated to determine the average viscosity.

### Examples

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The Example in Table 1 illustrates an example of a skin care composition that was made as follows: add the Water Phase ingredients to a suitable mixing container and mix until homogenous. Add the Oil Phase ingredients to a separate container and mix until homogenous. Add the Oil Phase to Water Phase and mill the mixture with a Tekmar® mill TK-25 or equivalent for 2 to 3 mins at 9000-11000 RPM. Replace the mill with a propeller mixer and add the remaining materials (e.g., pH adjuster, feel/appearance modifiers like starch and TiO₂) and/or phases, if present, to the composition one at a time and continue to mix until homogenous. Transfer the batch to final container.

TABLE 1
Example 1

| | Ex. 1 (wt %) |
|---|---|
| Water Phase | |
| USP Water | Q.S. |
| Water Soluble Actives | 0.005 |
| Polyacrylamide (and) C13-14 Isoparaffin | 2.2 |
| (and) Laureth-7¹ | |
| Acrylates/C10-30 Alkyl Acrylate | 0.15 |
| Crosspolymer² | |
| Disodium EDTA | 0.05 |
| Phenoxyethanol | 0.05 |
| Hydroxyacetophenone³ | 0.15 |
| Carnosine | 0.2 |
| Sodium Sulfite | 0.1 |
| Oil Phase | |
| Retinyl Propionate | 0.3 |
| Retinol | 0.0033 |
| Silicone Fluids | 18 |
| Silicone Elastomer | 10 |
| Laureth-4 | 0.2 |
| Remaining Materials | |
| Glycerin (99%) | 7 |
| Titanium Dioxide (75%) | 0.0875 |
| Modified Corn Starch⁴ | 5 |

### Suppliers for Table 1:

1. Sepigel™ 305 from Seppic® Corporation
2. Carbopol® Ultrez 21 polymer from Lubrizol®
3. Symsave® H from Symrise®
4. Pure-Dent® from GPC®

Table 2, below, includes Ex. A-H that include Aqual® or linear aldehydes with a mixture of chain lengths between 8-11 carbons in combination with Example 1 (see Table 1). The Aqual® (available from Mane®) is 6-cyclopentylidenehexanal. It was found that a very low level of Aqual® is needed to provide the malodor benefit and the in these examples Aqual® was delivered in a carrier fluid to help ensure homogeneous distribution throughout the composition. In Ex. A-D, the carrier fluid was dipropylene glycol. The carrier fluid is not believed to provide any malodor benefit.

TABLE 2
Example

| | |
|---|---|
| Control | Ex. 1 |
| Ex. A | Ex. 1 + 0.0025% Aqual ® + 0.0475% dipropylene glycol |
| Ex. B | Ex. 1 + 0.0015% Aqual ® + 0.0285% dipropylene glycol |
| Ex. C | Ex. 1 + 0.0005% Aqual ® + 0.0095% dipropylene glycol |
| Ex. D | Ex. 1 + 0.00025% Aqual ® + 0.00475% dipropylene glycol |
| Ex. E | +0.05% linear aldehydes with a mixture of chain lengths between 8-11 carbons |
| Ex. F | +0.03% linear aldehydes with a mixture of chain lengths between 8-11 carbons |
| Ex. G | +0.01% linear aldehydes with a mixture of chain lengths between 8-11 carbons |
| Ex. H | +0.005% linear aldehydes with a mixture of chain lengths between 8-11 carbons |

Table 3, below, shows the change in viscosity and pH of Ex. 1 (see Table 1) and Ex. A-H (see Table 2) at initial (i.e., soon after the sample was made) and at accelerated stability conditions of 2 weeks 60° C. and 1 month at 40° C./75% relative humidity After each example was made, it was put in a 2.5 oz glass jar with a lid. All of the samples were put into a large plastic container, which was put in the appropriate accelerated stability room and not exposed to light. The viscosity is measured according to the Viscosity Test Method, described herein. The viscosity of the example is considered acceptable and stable if it does not change by more than 30% between the initial value and the value at both accelerated stability conditions. The pH is acceptable and stable if it is greater than 5.

In Table 3, the viscosity and pH of Ex. A-G is compared to Ex. 1 (control). The viscosity values and pH values for Ex. A-G are similar to that of Ex. 1, and all of these examples fall within the acceptable range. This indicates that the additional aldehyde in the composition does not impact the stability of the viscosity and pH.

TABLE 3

| | Viscosity (cP) a | | | pH | | |
|---|---|---|---|---|---|---|
| | | 2 weeks at | 1 month at | | 2 weeks at | 1 month at |
| Example | Initial | 60° C. | 40° C. | Initial | 60° C. | 40° C. |
| Ex. 1 | 56867 | 50000 | 53400 | 6.35 | 5.8 | 5.9 |
| Ex. A | 55850 | 53266 | 52550 | 6.23 | 5.75 | 5.83 |
| Ex. B | 58200 | 57066 | 52000 | 6.14 | 5.65 | 5.86 |
| Ex. C | 55200 | 50600 | 49900 | 6.25 | 5.69 | 5.81 |
| Ex. D. | 55400 | 50600 | 50000 | 6.28 | 5.68 | 5.8 |
| Ex. E | 46800 | 49000 | 45600 | 6.25 | 5.75 | 5.91 |
| Ex. F | 47933 | 49050 | 44800 | 6.27 | 5.73 | 5.85 |
| Ex. G | 52000 | 46700 | 49000 | 6.32 | 5.79 | 5.82 |
| Ex. H | 51800 | 49133 | 48200 | 6.3 | 5.75 | 5.82 |

Table 4, below, shows the b-value and % retinyl propionate remaining at initial (i.e., soon after the sample was made) and at accelerated stability conditions. The b-value was determined by the Color Test Method, described herein. The % retinyl propionate remaining was determined according to the HPLC Test Method, described herein.

In Table 4, the retinyl stability and yellowness of Ex. A-G is compared to Ex. 1 (control). The % retinyl propionate remaining for Ex. A-G is close to that of Ex. 1, and all examples fall within the acceptable range. The b-value for Ex. A-G is also similar to Ex. 1 and the aldehyde does not exacerbate the yellowing of the product or have a substantial impact on pH. This indicates that the additional aldehyde in the composition does not impact the stability of the viscosity and pH.

TABLE 4

| | b-value | | | |
|---|---|---|---|---|
| | | 2 weeks at | 1 month at | % Retinyl Propionate |
| Example | Initial | 60° C. | 40° C. | Remaining |
| Ex. 1 | 2.15 | 9 | 4.1 | 90.0 |
| Ex. A | 1.33 | 7.65 | 4.04 | 83.3 |
| Ex. B | 2.52 | 8.9 | 4.21 | 90 |
| Ex. C | 2.32 | 9.32 | 3.5 | 90 |
| Ex. D. | 2.31 | 8.79 | 3.54 | 86.7 |
| Ex. E | 1.2 | 8.18 | 3.96 | 86.7 |
| Ex. F | 2.5 | 8.09 | 3.57 | 86.7 |
| Ex. G | 2.65 | 9.28 | 3.39 | 90 |
| Ex. H | 2.35 | 9.41 | 3.3 | 90 |

Samples of Ex. 1 and Ex. A-H were subjected to accelerated stability conditions by storing them for 2 weeks at a temperature of 60° C. These samples were then evaluated by three trained perfume experts who smelled the aged samples and provided their comments. The summarized evaluations are presented in Table 5, below. Based on the evaluations, it was found that Ex. A-H all exhibited an improvement over the control (Ex. 1). These examples effectively addressed the base odor and were potentially more acceptable to consumers compared to the control.

TABLE 5

| | Comments |
|---|---|
| Ex. 1 | Mild base odor |
| Ex. A | Covering base odor, detecting watery, ozonic notes from Aqual ® |
| Ex. B | Covering base odor, detecting watery, ozonic notes from Aqual ® |
| Ex. C | Covering base, but detect ozonic notes from Aqual ® |
| Ex. D | Covering base odor with a slight amount of Aqual ® noticeable |
| Ex. E | Covering base, smells aldehydic from accord |
| Ex. F | Covering base, smells aldehydic from accord |
| Ex. G | Covering base, but accord is still noticeable (aldehydic, slight citrus) |
| Ex. H | Covering base, but accord is still noticeable (aldehydic, slight citrus) |

The control and Examples X and Y, in Table 6, below, were made according to the same method used for the examples in Table 1. A test was run with 10 participants who were blinded and 0.1g of the control and Example X and Y was applied to the inside of one of their forearms. Each participant was asked to rate the odor intensity from a 0-10 scale (0 being no odor, 10 being strong odor) immediately after application (initial timepoint), 1.5 minutes after application, and 3 minutes after application.

| | | Timepoint | | |
|---|---|---|---|---|
| | | | 1.5 minutes after | 3 minutes after |
| Example | Composition | Initial | application | application |
| Control | Ex. 1 | 3.7 | 1.8 | 1.2 |
| Ex. X | Ex. 1 + 0.0001% | 2.55 | 1.05 | 0.8 |
| | Aqual ® + | | | |
| | 0.0019% | | | |
| | dipropylene | | | |
| | glycol | | | |
| Ex. Y | Ex. 1 + 0.0005% | 3.9 | 3.75 | 1.85 |
| | Aqual ® + | | | |
| | 0.0095% | | | |
| | dipropylene | | | |
| | glycol | | | |

As shown in Table 6, both Examples X and Y, which contained Aqual®, had a lower odor intensity at all three timepoints, as compared to the control. While both Examples X and Y are likely acceptable to consumers, Example X may be preferred because it exhibits less raw material (Aqual®) scent.

The Examples in Table 7 could be made according to the same method used for the example in Table 1.

TABLE 7

| | Ex. 2 (wt %) | Ex. 3 (wt. %) | Ex. 4 (wt. %) |
|---|---|---|---|
| Water Phase | | | |
| USP Water | Q.S. | Q.S. | Q.S. |
| Water Soluble Actives | 0.005-1 | 0.005-1 | 0.005-1 |
| Polyacrylamide (and) C13-14 | 1.5-2.6 | 1.5-2.6 | |
| Isoparaffin (and) Laureth-7¹ | | | |
| Acrylates/C10-30 Alkyl | 0.1-0.3 | 0.1-0.3 | |
| Acrylate Crosspolymer² | | | |
| Microcrystalline Cellulose (and) | | | 0.5-1 |
| Sphingomonas Ferment Extract | | | |
| (and) Cellulose Gum | | | |
| Disodium EDTA | 0.05 | 0.05 | 0.05 |
| Phenoxyethanol | 0.05-0.2 | 0.05-0.2 | 0.05-0.2 |
| Hydroxyacetophenone³ | 0.05-0.2 | 0.05-0.2 | 0.05-0.2 |
| Carnosine | 0.005-0.2 | 0.005-0.2 | 0.005-0.2 |
| Sodium Sulfite | 0.05-0.2 | 0.05-0.2 | 0.05-0.2 |
| Oil Phase | | | |
| Retinyl Propionate | 0.3-0.4 | 0.3-0.4 | |
| Retinol | 0.0033 | 0.0033 | 0.5-1 |
| Silicone Oils | 14-18 | 14-18 | |
| Silicone Elastomer | 4-10 | 4-10 | |
| Coco-Caprylate/Caprate | | | 7-9 |
| Laureth-4 | 0.2 | 0.2 | 0-0.2 |
| Polysorbate-20 | | | 0-0.2 |
| Remaining Materials | | | |
| Glycerin (99%) | 3-7 | 3-7 | 3-7 |
| Titanium Dioxide (75%) | 0-0.1 | 0-0.1 | |
| Modified Corn Starch⁴ | 2-5 | 2-5 | |
| 6-cyclopentylidenehexanal⁵ | 0.0001-0.0025 | | 0.0001-0.0025 |
| Dipropylene glycol | 0.00475-0.0475 | | 0.00475-0.0475 |
| linear aldehydes with a mixture | | 0.0001-0.05 | |
| of chain lengths between 8-11 | | | |
| carbons | | | |
| Fragrance | 0-0.05% | 0-0.05% | 0-0.05% |

### Suppliers for Table 1 and Table 6:

1. Sepigel™ 305 from Seppic® Corporation
2. Carbopol® Ultrez 21 polymer from Lubrizol®
3. Symsave® H from Symrise®
4. Pure-Dent® from GPC®
5. Aqual® (available from Mane®)

### Combinations:

A. A skin care composition having reduced malodor comprising:
   a. an aldehyde comprising an unsaturated cyclic aldehyde with 11 carbon atoms;
   b. a retinoid;
   c. a dermatologically acceptable carrier.
B. A skin care composition having reduced malodor comprising:
   a. an aldehyde comprising a mixture of linear aldehydes comprising:
      i. a first linear aldehyde comprising a chain length of about 4 to about 20 carbon atoms, preferably from 6 to 16 carbon atoms, more preferably from 7 to 14 carbon atoms, and more preferably from 8 to 11 carbon atoms;
      ii. a second linear aldehyde comprising a chain length of about 4 to about 20 carbon atoms, preferably from 6 to 16 carbon atoms, more preferably from 7 to 14 carbon atoms, and more preferably from 8 to 11 carbon atoms;
   wherein the chain length of the first aldehyde and the second aldehyde are different;
   b. a retinoid;
   c. a dermatologically acceptable carrier.
C. The skin care composition according to Paragraphs A-B, wherein the composition comprises from about 0.00005% to about 0.05% of the aldehyde, preferably about 0.000075% to about 0.0025%, more preferably from about 0.0001% to about 0.0015%, and even more preferably about 0.0001% to about 0.0005%.
D. The skin care composition according to Paragraph A and C, wherein the composition comprises from about 0.00005% to about 0.015%, preferably from about 0.0001% to about 0.01%, preferably from about 0.0001% to about 0.001%, more preferably from about 0.0002% to about 0.0005%, more preferably from about 0.00025% to about 0.0025%, and even more preferably from about 0.0002% to about 0.002% of the unsaturated cyclic aldehyde with 11 carbon atoms.
E. The skin care composition according to Paragraphs A and C-D, wherein the unsaturated cyclic aldehyde comprises carbon atoms are in both cyclic and linear configuration.
F. The skin care composition according to Paragraph E, wherein the linear configuration comprises from about 7 to about 20 carbon atoms, preferably from about 9 to about 15 carbon atoms, more preferably from about 10 to about 13 carbon atoms, and even more preferably 5 to about 7 carbon atoms.
G. The skin care composition according to Paragraphs E-F, wherein the linear configuration comprises at least one degree of unsaturation, preferably exactly one degree of unsaturation.
H. The skin care composition according to Paragraph A and C-G, wherein the unsaturated cyclic aldehyde comprises 6-cyclopentylidenehexanal.
I. The skin care composition according to Paragraphs B-C, wherein the composition comprises from about 0.0001% to about 0.05%, preferably from about 0.0003% to about 0.03%, more preferably from about 0.0004% to about 0.02%, and even more preferably from about 0.0005% to about 0.01% of the mixture of linear aldehydes.
J. The skin care composition according to Paragraphs B-C and I, wherein the mixture of linear aldehydes comprises ≥10%, by weight of the mixture, preferably ≥15%, by weight of the mixture, more preferably ≥17%, by weight of the mixture, and even more preferably ≥20%, by weight of the mixture of the first linear aldehyde and the second linear aldehyde.
K. The skin care composition according to Paragraphs B-C and I-J, wherein the weight ratio of the first linear aldehyde to the second linear aldehyde is from about 1:2 to about 2:1, preferably from about 2:3 to about 4:2, more preferably from about 3:4 to about 4:3, and even more preferably from about 4:5 to about 5:1.
L. The skin care composition according to Paragraphs B-C and I, wherein the mixture of linear aldehydes further comprises a third linear aldehyde comprising a chain length of about 4 to about 20 carbon atoms, preferably from 6 to 16 carbon atoms, more preferably from 7 to 14 carbon atoms, and more preferably from 8 to 11 carbon atoms;
wherein the chain length of the third linear aldehyde is different than the chain length of the first and the second linear aldehydes.
M. The skin care composition according to Paragraph L, wherein the mixture of linear aldehydes comprises ≥10%, by weight of the mixture, preferably ≥15%, by weight of the mixture, more preferably ≥17%, by weight of the mixture, and even more preferably ≥20%, by weight of the mixture of the third linear aldehyde.
N. The skin care composition according to Paragraphs L-M, wherein the weight ratio of the first linear aldehyde to the third linear aldehyde is from about 1:2 to about 2:1, preferably from about 2:3 to about 4:2, more preferably from about 3:4 to about 4:3, and even more preferably from about 4:5 to about 5:1.
O. The skin care composition according to Paragraphs L-N, wherein the carbon chain of the third linear aldehyde is saturated.
P. The skin care composition according to Paragraphs L-O, wherein the mixture of linear aldehydes further comprises a fourth linear aldehyde comprising a chain length of about 4 to about 20 carbon atoms, preferably from 6 to 16 carbon atoms, more preferably from 7 to 14 carbon atoms, and more preferably from 8 to 11 carbon atoms;
wherein the chain length of the fourth linear aldehyde is different than the chain length of the first, second, and third linear aldehydes.
Q. The skin care composition according to Paragraph P, wherein the mixture of linear aldehydes comprises ≥10%, by weight of the mixture, preferably ≥15%, by weight of the mixture, more preferably ≥17%, by weight of the mixture, and even more preferably ≥20%, by weight of the mixture of the fourth linear aldehyde.
R. The skin care composition according to Paragraph P-Q, wherein the weight ratio of the first linear aldehyde to the fourth linear aldehyde is from about 1:2 to about 2:1, preferably from about 2:3 to about 4:2, more preferably from about 3:4 to about 4:3, and even more preferably from about 4:5 to about 5:1.
S. The skin care composition according to Paragraphs L-R, wherein the carbon chain of the fourth linear aldehyde is saturated.
T. The skin care composition according to Paragraph B-C and I-S, wherein the carbon chain of the first linear aldehyde and/or the second linear is saturated.
U. The skin care composition according to Paragraphs A-T, wherein the composition is free of DMDM hydantoin, imadazolidinyl urea, diazlidinyl urea, sodium hydroxyl, and methyl glycinate.
V. The skin care composition according to Paragraphs A-U, wherein the composition further comprises a preservative system comprising hydroxyacetophenone; carnosine; and sodium sulfite.
W. The skin care composition according to Paragraphs A-V, further comprising one or more chelating agents selected from disodium EDTA, tetrasodium EDTA, tetrahydroxypropyl ethylenediamine, etidronic acid, sodium phytate, phytic acid, oxalic acid and derivatives, sodium gluconate, trisodium ethylenediamine disuccinate, or mixtures thereof.
X. The skin care composition according to Paragraphs A-W, wherein the retinoid is selected from retinyl palmitate, retinyl acetate, retinyl propionate, or mixtures thereof.
Y. The skin care composition according to Paragraphs A-X, wherein the skin care composition is free of formaldehyde and formaldehyde-releasing agents.
Z. The skin care composition according to Paragraphs A-Y, wherein the skin care composition is free of parabens and sulfate-based surfactants.
AA. The skin care composition according to Paragraphs A-Z, wherein the skin care composition comprises a b-value of less than 25, preferably less than 20, more preferably less than 14, and even more preferably less than or equal to 10 according to the Color Test Method, described herein.
BB. Use of the skin care composition according to any preceding claim, to provide a retinol containing composition with reduced malodor.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition having reduced malodor comprising:
a. an unsaturated cyclic aldehyde with at least 11 carbon atoms;
b. a retinoid;
c. a dermatologically acceptable carrier.

2. The composition of claim 1, wherein the composition comprises from about 0.00005% to about 0.005% of the unsaturated cyclic aldehyde.

3. The composition of claim 2, wherein the unsaturated cyclic aldehyde comprises carbon atoms are in both cyclic and linear configuration.

4. The composition of claim 3, wherein the unsaturated cyclic aldehyde comprises 6-cyclopentylidenehexanal.

5. The skin care composition of claim 1, wherein the composition is free of DMDM hydantoin, imadazolidinyl urea, diazlidinyl urea, sodium hydroxyl, and methyl glycinate.

6. The skin care composition of claim 5, wherein the composition further comprises a preservative system comprising hydroxyacetophenone; carnosine; and sodium sulfite.

7. The skin care composition of claim 1, further comprising one or more chelating agents chosen from disodium EDTA, tetrasodium EDTA, tetrahydroxypropyl ethylenediamine, etidronic acid, sodium phytate, phytic acid, oxalic acid and derivatives, sodium gluconate, trisodium ethylenediamine disuccinate, or mixtures thereof.

8. The skin care composition of claim 1, wherein the retinoid is chosen from retinyl palmitate, retinyl acetate, retinyl propionate, or mixtures thereof.

9. A skin care composition having reduced malodor comprising:
a. a mixture of linear aldehydes comprising:
i. a first linear aldehyde comprising a chain length of about 4 to about 20 carbon atoms;
ii. a second linear aldehyde comprising a chain length of about 4 to about 20 carbon atoms; wherein the chain length of the first aldehyde and the second aldehyde are different;
b. a retinoid;
c. a dermatologically acceptable carrier.

10. The composition of claim 9, wherein the composition comprises from about 0.00005% to about 0.05% of the mixture of linear aldehydes.

11. The skin care composition of claim 9, wherein the mixture of linear aldehydes further comprises a third linear aldehyde comprising a chain length of about 4 to about 20 carbon atoms; wherein the chain length of the third linear aldehyde is different than the chain length of the first and the second.

12. The skin care composition of claim 9, wherein the first linear aldehyde and the second linear aldehyde comprise a carbon chain length of about 8 to about 11 carbon atoms.

13. The skin care composition of claim 9, wherein the mixture of linear aldehydes comprises ≥10%, by weight of the mixture, of the first linear aldehyde and ≥10%, by weight of the mixture, of the second linear aldehyde.

14. The skin care composition of claim 9, wherein the retinoid is chosen from retinyl palmitate, retinyl acetate, retinyl propionate, or mixtures thereof.

15. The skin care composition of claim 9, wherein the skin care composition is free of parabens, sulfate-based surfactants, formaldehyde, and formaldehyde-releasing agents.

16. A skin care composition having reduced malodor comprising:
a. a malodor reducing material comprising an aldehydic moiety;
b. a retinoid;
c. a dermatologically acceptable carrier.

17. The skin care composition of claim 16, wherein the composition comprises from about 0.00005% to about 0.001% of the malodor reducing material.

18. The skin care composition of claim 16, wherein the malodor reducing material is unsaturated.

19. The skin care composition of claim 18, wherein the aldehyde comprises a cyclic aldehyde.

20. The skin care composition of claim 18, wherein the unsaturated cyclic aldehyde comprises 11 carbon atoms.
